(19) United States
(12) Patent Application Publication
(10) Pub. No.: US 2017/0209316 A1
(43) Pub. Date: Jul. 27, 2017

(54) METHODS AND ARRANGEMENTS IN A GARMENT WITH FLAPS

(71) Applicant: Jill BERKLEY, Belvidere, IL (US)

(72) Inventor: Jill BERKLEY, Belvidere, IL (US)

(21) Appl. No.: 15/005,025

(22) Filed: Jan. 25, 2016

(51) Int. Cl. A61F 15/00

(52) U.S. Cl. CPC A61F 15/008

(57) ABSTRACT

Disclosed are the method and an arrangement incorporated with a garment for eliminating the friction to an incision area of a recipient of the surgery. Further the arrangement allowing easy access to the incision area. The arrangement includes one or more flaps, one or more holding units, and one or more closing units. The flaps are coupled with the garment to covering the incision area of the recipient of the surgery having plurality of openable ends. The openable ends include a top upper end, a first side end, and a bottom lower end. The holding unit supports the flap with the garment from a closed second side. The second side is the opposite of the first side. The closing unit secures the flap from the plurality of the openable ends.

### FIELD OF THE PRESENT INVENTION

The presently disclosed embodiments are related, in general, to a garment or garments. More particularly, the presently disclosed embodiments are related to methods and arrangements for eliminating the friction to an incision area of a recipient of surgery.

### BACKGROUND OF THE PRESENT INVENTION

Various surgeries such as knee replacements, heart surgery, or hip replacements have emerged as a routine surgery in the world. The recipients of the knee replacement surgery are usually older and have arthritic joints. However, an increasing number of younger people are also replacing their knees because of injury from high-impact sports and activities that wear out their knees early. Further, there are various problems only recipients with knee injuries understand, such as unable to walk properly; showering becomes a painful activity; uncomfortable sleeping positions etc.

The incision or scar is uncomfortable for the recipient of the knee replacement surgery, especially when the pants or blanket are rubbing on the incision. The incisions can make it very uncomfortable for the recipient to wear long pants. Thus, a need exists to make an arrangement in a garment to protect the incision, eliminate the friction to an incision area, and further allow access to the incision area.

Further limitations and disadvantages of conventional and traditional approaches will become apparent to one of skill in the art, through comparison of described systems with some aspects of the present disclosure, as set forth in the remainder of the present application and with reference to the drawings.

### SUMMARY OF THE PRESENT INVENTION

A method and an arrangement incorporated with a garment for eliminating the friction to an incision area of a recipient of the surgery are provided substantially as shown in, and/or described in connection with, at least one of the figures, as set forth more completely in the claims.

According to embodiments illustrated herein, there is provided an arrangement incorporated with a garment for eliminating the friction to an incision area of a recipient of the surgery, and further allowing access to the incision area.

The arrangement includes one or more flaps, one or more holding units, and one or more closing units. The flaps are coupled with the garment to covering the incision area of the recipient of the surgery having plurality of openable ends. The openable ends include a top upper end, a first side end, and a bottom lower end. The holding unit supports the flap with the garment from a closed second side. The second side is the opposite of the first side. The closing unit secures the flap from the plurality of the openable ends.

The closing unit includes at least one of a zip fastener; one or more buttons; and a Velcro. The one or more flaps are detachable from the garment. The one or more flaps are made up from at least one of: foam, cotton, and polyester. The surgery is at least one of: knee replacement surgery, heart surgery, and hip replacement surgery. The one or more flaps are at least of: rectangular shape, square shape, and semicircular shape.

According to embodiments illustrated herein there is provided a method for eliminating the friction to an incision area of a recipient of the surgery, and further allowing easy access to the incision area through an arrangement incorporated with a garment. The method includes the step of covering the incision area of the recipient of the surgery having plurality of openable ends through one or more flaps coupled with the garment. In an embodiment, the openable ends include a top upper end, a first side end, and a bottom lower end. Further, the method includes the step of supporting the flap with the garment from a closed second side through one or more holding units, wherein the second side is the opposite of the first side. Thereafter, the method includes the step of securing the flap from the plurality of the openable ends through one or more closing unit.

These and other features and advantages of the present disclosure may be appreciated from a review of the following detailed description of the present disclosure, along with the accompanying figures in which like reference numerals refer to like parts throughout.

### BRIEF DESCRIPTION OF DRAWINGS

The accompanying drawings illustrate the various embodiments of arrangements, methods, and other aspects of the disclosure. Any person with ordinary skills in the art will appreciate that the illustrated element boundaries (e.g., boxes, groups of boxes, or other shapes) in the figures represent one example of the boundaries. In some examples, one element may be designed as multiple elements, or multiple elements may be designed as one element. In some examples, an element shown as an internal component of one element may be implemented as an external component in another, and vice versa. Further, the elements may not be drawn to scale.

Various embodiments will hereinafter be described in accordance with the appended drawings, which are provided to illustrate and not to limit the scope in any manner, wherein similar designations denote similar elements, and in which:

FIG. 1 illustrates an arrangement incorporated with a garment for eliminating the friction to an incision area, in accordance with at least one embodiment; and

FIG. 2 illustrates a flow chart of a method for eliminating the friction to an incision area, in accordance with at least one embodiment.

### DETAILED DESCRIPTION OF THE DRAWINGS

The present disclosure is best understood with reference to the detailed figures and description set forth herein. Various embodiments are discussed below with reference to the figures. However, those skilled in the art will readily appreciate that the detailed descriptions given herein with respect to the figures are simply for explanatory purposes as the methods and systems may extend beyond the described embodiments. For example, the teachings presented and the needs of a particular application may yield multiple alternative and suitable approaches to implement the functionality of any detail described herein. Therefore, any approach may extend beyond the particular implementation choices in the following embodiments described and shown.

References to “one embodiment,” “at least one embodiment,” “an embodiment,” “one example,” “an example,” “for example,” and so on indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” does not necessarily refer to the same embodiment.

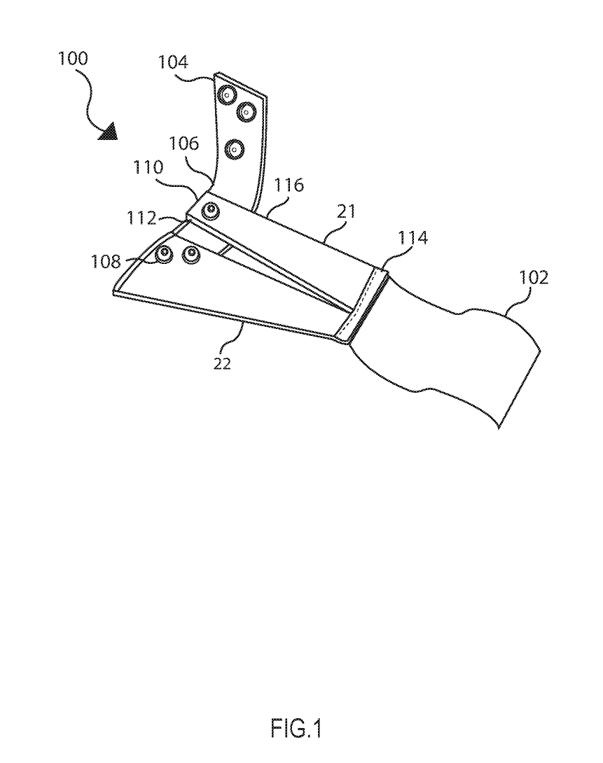

FIG. 1 is an arrangement 100 incorporated with a garment for eliminating the friction to an incision area, in which various embodiments may be implemented.

The arrangement 100 incorporated with a garment 102 for eliminating the friction to an incision area of a recipient of the surgery. Further, the arrangement 100 allows easy access to the incision area. The arrangement 100 includes one or more flaps 104, one or more holding units 106, and one or more closing units 108. The flaps 104 are coupled with the garment 102 to covering the incision area of the recipient of the surgery having plurality of openable ends. The openable ends include a top upper end 110, a first side end 112, and a bottom lower end 114. The holding unit 106 supports the flap 104 with the garment 102 from a closed second side 116. The second side 116 is opposite to the first side 112. The closing unit 108 secures the flap 104 from the plurality of the openable ends.

The closing unit 108 includes at least one of: a zip fastener; one or more buttons; and a Velcro. A Velcro hook or loop fastener attachment or a press-stud attachment may be used as the closing unit 108. However, it will be readily apparent to those skilled in the art that various other types of closing unit 108 may be configured with garment 102.

The garment 102 is at least one of: a pair of pants; and a blanket. For instance, a pair of pants with a knee flap may open over the incision caused by knee surgery. Similar arrangement of a flap may be integrate with a blanket because the incision is sensitive to the things rubbing on the incision. The flap gives the recipient relief.

The one or more flaps 104 are detachable from the garment. A person skilled in the art will understand that the scope of the disclosure is not limited to the surgeries such as knee surgery, hip surgery, or heart surgery. In an embodiment, the arrangement 100 may be realized in injuries caused by accidents or fractures without departing from the spirit of the disclosure.

The one or more flaps 104 are made up from at least one of: foam; cotton; and polyester. The pants should be a soft comfortable material, like pajama pants or sweat pants, and may be a more formal pair, like slacks.

The surgery is at least one of: knee replacement surgery; heart surgery; and hip replacement surgery. For example, the pants made to have a flap over either the left knee, right knee, or both hip areas. Similarly, the blanket may have the similar flaps for the person with heart surgery, a blanket with a heart shaped patch in the center that may be buttoned open.

The one or more flaps 104 are at least of: rectangular shape; square shape; and semicircular shape. However, it will be readily apparent to those skilled in the art that various other shapes of flaps 104 may be configured according to the shape and size of the garment 102.

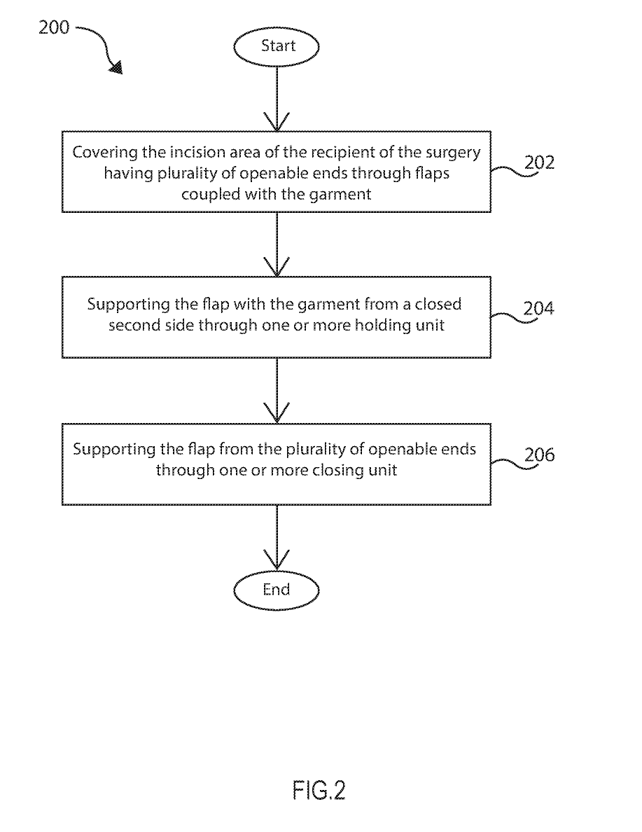

FIG. 2 illustrates a flow chart of a method 200 for eliminating the friction to an incision area, in accordance with at least one embodiment. The method 200 for eliminating the friction to an incision area of a recipient of the surgery, and further allowing easy access to the incision area through an arrangement incorporated with a garment. The method 200 of the present invention initiates with a step 202 of covering the incision area of the recipient of the surgery having plurality of openable ends through one or more flaps coupled with the garment. In an embodiment, the openable ends include a top upper end, a first side end, and a bottom lower end.

The step 202 is then followed by a step 204 of supporting the flap with the garment from a closed second side through one or more holding units. In an embodiment, the second side is the opposite of the first side. The step 204 is then followed by a step 206 of securing the flap from the plurality of the openable ends through one or more closing unit.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. An arrangement incorporated with a garment for eliminating the friction to an incision area of a recipient of the surgery, and further allowing access to the incision area, the arrangement comprising:
one or more flaps coupled with the garment to covering the incision area of the recipient of the surgery having plurality of openable ends, wherein the openable ends include a top upper end, a first side end, and a bottom lower end;
one or more holding units to support the flap with the garment from a closed second side, wherein the second side is the opposite of the first side; and
one or more closing unit to secure the flap from the plurality of the openable ends.

2. The arrangement according to claim 1, wherein the closing unit includes at least one of: a zip fastener; one or more buttons; and a Velcro.

3. The arrangement according to claim 1, wherein the garment is at least one of: a pair of pants and a blanket.

4. The arrangement according to claim 1, wherein the one or more flaps are detachable from the garment.

5. The arrangement according to claim 1, wherein the one or more flaps are made up from at least one of: foam; cotton; and polyester.

6. The arrangement according to claim 1, wherein the surgery is at least one of: knee replacement surgery; heart surgery; and hip replacement surgery.

7. The arrangement according to claim 1, wherein the one or more flaps are at least of: rectangular shape; square shape; and semicircular shape.

8. A method for eliminating the friction to an incision area of a recipient of the surgery, and further allowing easy access to the incision area through an arrangement incorporated with a garment, the method comprising the steps of:
covering the incision area of the recipient of the surgery having plurality of openable ends through one or more flaps coupled with the garment, wherein the openable ends include a top upper end, a first side end, and a bottom lower end;
supporting the flap with the garment from a closed second side through one or more holding units, wherein the second side is the opposite of the first side; and
securing the flap from the plurality of the openable ends through one or more closing unit.

9. The method of claim 8, wherein the closing unit includes at least one of: a zip fastener; one or more buttons; and a Velcro.

10. The method of claim 8, wherein the garment is at least one of: a pair of pants and a blanket.

11. The method of claim 8, wherein the one or more flaps are detachable from the garment.

12. The method of claim 8, wherein the one or more flaps are made up from at least one of: foam; cotton; and polyester.

13. The method of claim 8, wherein the surgery is at least one of: knee replacement surgery; heart surgery; and hip replacement surgery.

14. The method of claim 8, wherein the one or more flaps are at least of: rectangular shape; square shape; and semicircular shape.
